(19) United States
(12) Patent Application Publication
(10) Pub. No.: US 2004/0142316 A1
(43) Pub. Date: Jul. 22, 2004

(54) Analytical test approach for blood

(76) Inventors: Richard Owen, Melbourn Hertfordshire (GB); Kevin Clarke, Melbourn (GB)

Correspondence Address: NIXON & VANDERHYE, PC, 1100 N GLEBE ROAD, 8TH FLOOR, ARLINGTON, VA, 22201-4714, US

(21) Appl. No.: 10/476,008

(22) PCT Filed: Apr. 22, 2002
(86) PCT No.: PCT/GB02/01922

(30) Foreign Application Priority Data
Apr. 25, 2001 (GB) 0110172.4

(51) Int. Cl. A01N 1/02; C12Q 1/04

(52) U.S. Cl. 435/2; 435/34

(57) ABSTRACT

The invention provides a method of testing for a blood-borne agent present in or on the surface of white blood cells or on platelets, comprising: (a) providing a blood-derived sample enriched in such white blood cells and/or platelets: and (b) testing for the presence or absence, or amount or concentration, of said agent in said sample.

### FIELD OF THE INVENTION

The invention relates to the analytical testing of blood, especially for disease agents. It finds particular utility in the screening of human blood samples donated for transfusion purposes for prion-mediated diseases, particularly new variant Creutzfeldt-Jakob disease (vCJD).

### BACKGROUND OF THE INVENTION

The awareness of the risks of disease transmission through contaminated blood and blood products has in recent years lead to greater emphasis upon testing for viral, bacterial and prion diseases, especially in donated blood. With donated blood, e.g. in blood banks, these tests are routinely applied in an attempt to isolate contamination before processing takes place to minimise the risks of disease transmission. Analytical tests exist to identify many viral, bacterial and prion diseases of general concern.

Many current tests rely on the detection of antibodies to disease agents. Such tests are carried out on blood plasma Others rely on the detection of nucleic acids characteristic of the agents, notably viral DNA sequences, and are also usually carried out on plasma or serum.

However, one factor that makes many of these tests time consuming and expensive is the need to pre-concentrate the active agent by chemical, physical or microbiological means to a level that will give a reasonably clear positive/negative response. These concentration/amplification techniques are often complex, time-consuming, prone to error/cross contamination and frequently require a relatively large sample to achieve a high level of confidence in the result.

With prion-modified diseases in particular the size of blood sample necessary to ensure sufficient suspect prion material will be captured can be extremely large. Currently prion diseases such as vCJD are of major concern and to reduce the likelihood of contaminated blood products in some countries such as the USA individuals known to have spent time in a country where vCJD is present are excluded from donating blood. With frequent overseas travel becoming more commonplace this inevitably will begin to have a significant effect upon the availability of suitable donated blood. The alternative approach of testing carries with it some risk, and also requires a large proportion of the donated blood to be used in the test process to ensure accuracy, thus considerably reducing the available blood for products.

Prion-mediated diseases are of great public concern. Various methods have been proposed for their diagnosis. For example, Boehringer Ingelheim's WO00/65357 provides a test method suitable for clinical or pre-clinical diagnosis of transmissible spongiform encephalopathies (TSES) such as BSE by concentrating target cells and analysing them for the presence of a marker protein for the TSE. However, amongst other drawbacks, this method requires that whole cells be obtained and that extensive laboratory handling procedures be carried out on them, and is limited because it detects a marker protein rather than the infectious agent itself. It is therefore unsuitable for large-scale screening of blood donations. There presently exists no reliable, straightforward, blood-based test for vCJD in the blood supply. The only reliable ways to assess the vCJD state of a patient are to monitor symptoms and examine samples of brain tissue for the characteristic spongiform morphology associated with the disease, and with animal prion-mediated diseases such as scrapie in sheep and bovine spongiform encephalomyelitis (BSE) in cattle. This cannot be done on a living patient and is therefore not of any use in blood screening or diagnosis. Moreover, although symptomatic individuals will evidently be excluded from blood donation programmes, many sufferers are likely to be asymptomatic and thus prima facie able to donate blood. vCJD is not thought to be a common disease. It is therefore very crude to exclude individuals on large-scale bases such as geographical origin, as is being done in the USA at the moment, as it inevitably excludes many individuals that do not carry the disease. Nevertheless, the seriousness of vCJD as a disease means that it is highly undesirable to have to ignore it in blood donation programmes.

A blood-based test for vCJD would thus be of great benefit in such programmes. It would improve both safety and public confidence in the blood supply, and also prevent the unnecessary exclusion of individuals from donation programmes with the result that more blood donors became available.

### SUMMARY OF THE INVENTION

It is observed that by nature, the white blood cells (leukocytes) contained in blood will envelop foreign agents by phagocytosis and start the process of destruction. In addition in the case of vCJD, the scrapie form of the prion (Sc) can attach to the normal (C) form of the prion on the surface of the white blood cells. It is a further observation that over the past few years the technology of white cell filtration from blood has become well developed (see Asahi Medical Co's EP-A-0 502,213 for example). In most developed countries, white blood cells are routinely filtered from donated blood, and other countries are likely to follow. In a typical filtration process, almost all of the white cells from a unit of donated blood will be captured along with the active agents therein and remain in the filter unit with very small quantities of whole blood. Platelets are also concentrated into such filtered samples.

It has been proposed to remove white blood cells by leuko-filtration from blood products for transfusion in order to reduce the risk of transmission of transmissible spongiform encephalopathies (TSEs) (see Chapman et al's U.S. Pat. No. 6,197,207), although the safety of “cleaning” blood in this manner must be questionable from the point of view of supply of blood products.

However, the concentrated white blood cell samples captured by such leuko-reducing filters are currently considered to be a waste product and disposed of. As discussed above, analytical tests are typically carried out on plasma. According to the invention, such concentrated white blood cell samples and platelet are put to advantageous use in analytical testing, because they act as concentrated reservoirs of blood-borne agents enveloped by, or attached to, the white blood cells and/or platelets. This is especially advantageous for prion-mediated diseases because such diseases currently require large blood sample sizes to test for.

Thus, the invention provides a means for testing for blood-borne agents, especially prions such as the scrapie. (Sc) prion characteristic of vCJD, whereas no such means are available at the present time. Advantageously, no wastage of blood is required, as the test is carried out on a blood component that is routinely discarded. This method also therefore fits neatly into existing blood donation, filtration and testing procedures.

Accordingly, the invention provides:

a method of testing for a blood-borne agent present in or on the surface of white blood cells or in or on the surface of platelets, comprising: (a) providing a blood-derived sample enriched in such white blood cells and/or platelets; and (b) testing for the presence or absence, or amount or concentration, of said agent in said sample.

The invention also provides:

use of a blood-derived sample enriched in white blood cells and/or platelets in a method of testing for a blood-borne agent present or suspected of being present in or on the surface of said white blood cells and/or platelets.

The invention also provides:

a kit for carrying out a testing method according to the invention comprising a reagent or reagents capable of detecting said agent, and optionally a leuko-reducing filter unit and/or a reader capable of visualising the reagent or reagents following detection of the test agent.

### BRIEF DESCRIPTION OF THE DRAWINGS

FIG. 1: CJD Blood test on concentrated white blood cells: test principle.

(A) White blood cells and platelets contained in leukocyte reduction filter following conventional donation process.

(B) Lysis solution added to release prions contained in leukocytes.

(C) Filter saturated with:

plasminogen bound on the surface of magnetic beads

fluorescein-labelled antibody to PrP (which binds to both PrPc and PrPsc).

(D) Magnetic beads, now with PrPsc attached, are retained and observed by presence of fluorescein, unattached fluorescein rinsed away.

FIG. 2: Two possible practical embodiments of the invention.

Left-hand side (preferred): donated blood flows form the container at the top, down a tube and through a leuko-reducing filter (L), which removes a high proportion of the white blood cells and platelets and carries out leuko-reduction to the extent needed for blood safety, as well as acting as a concentrated source of white blood cells for use in the invention.

Right-hand side: As left-hand side but blood flows first through a preliminary leuko-reducing filter which removes a proportion of the white blood cells and serves as a concentrated source of white blood cells and/or platelets for use in the invention, then through a separate filter that carries out further leuko-reduction to the extent needed for blood safety.

FIGS. 3-7: Schematic depiction of a preferred testing method of the invention.

FIG. 3: (A) detachment of leuko-reducing filter, (B) white blood cells and/or platelets recovered therefrom, (C) white blood cell having on its surface normal (C) prions and vCJD disease agent prions (Sc).

FIG. 4: (A) Injection of reagent/lysis solution into the detached filter, (B) contacting (and lysis—not shown) of cells with plasminogen (P) bound to magnetic beads and fluorescein-labeled antibodies (F).

FIG. 5: Agitation of filter, lysis of cells, binding of Sc prions to plasminogen and antibodies, binding of C prion to antibodies only.

FIG. 6: Separation of magnetised, plasminogen-bound and antibody/fluorescein-labeled Sc prions, washing of filter to remove other components, including unmagnetised C prions.

FIG. 7: Visualisation of separated, antibody/fluorescein-labeled Sc prions via the fluorescein label.

FIG. 8: In-line detection of Sc prions according to the invention, via antibodies specific to the Sc prion.

(A, left panel): In-line arrangement of leukocyte-reduction filter with blood sample and collected leukocyte-reduced blood. A blood sample is provided in input bag 1. Blood flows into assay puck 2 as shown by arrow 5. 6 denotes a large pore size mesh; 7 denotes a port for reagents to the introduced and solutions drained out; 8 denotes a white blood cell/platelet mesh, and 10 denotes a screw-off test tube. Blood flows out of the assay puck as shown by arrow 9. Thus, the blood passes both through coarse mesh 6 and fine mesh 8. It then drains through leukocyte filter 3 into output bag 4. This results in white blood cells and platelets being collected in assay puck 2, prior to further filtration by filter 3.

(A, right panel): Antibodies specific to Sc prion are introduced into the puck, which contains concentrated white blood cells and platelets. The tubing at the top and bottom of the assay puck is clamped. Side port 7 is opened and, for example, 20 ml detergent and antibody-coated capture beads are added. Port 7 is resealed. The antibodies bind to Sc prions but not to C prions.

(B, left panel): The puck is agitated gently for, for example, 30 minutes. The puck is inverted and the clamps are opened to allow the contents to drain through coarse mesh 6. Antibody-coated capture beads, to which Sc prions are bound, remain behind. C Prions are not bound and thus drain away.

(B right panel): The puck is turned through 90 degrees to allow the beads to fall into test tube 10. Prion-releasing buffer, desirably 200 μl is added through side port 7 and the buffer and beads are incubated for, e.g. 5 minutes. The releasing buffer may be, for example, a concentrated salt solution, a strong acid or a strong base.

(C, upper drawing): Once prions have been released from the beads, the prion solution is extracted from the test tube, leaving the beads behind.

(C, lower drawing): The prion-containing solution is then transferred to a microtitre plate well for analysis.

FIG. 9: In-line detection of Sc prions according to the invention via antibodies binding to both Sc and C prions, with proteinase K digestion of C prions.

(A, left panel): In-line arrangement of leukocyte-reduction filter with blood sample and collected leukocyte-reduced blood. A blood sample is provided in input bag 1. Blood flows into assay puck 2 as shown by arrow 5. 6 denotes a large pore size (e.g. 1 to 3 mm) mesh; 7 denotes a port for reagents to the introduced and solutions drained out; 8 denotes a white blood cell/platelet mesh, and 10 denotes a screw-off test tube. Blood flows out of the assay puck as shown by arrow 9. Thus, the blood passes both through coarse mesh 6 and fine mesh 8. It then drains through leukocyte filter 3 into output bag 4. This results in white blood cells and platelets being collected in assay puck 2, prior to further filtration by filter 3.

(A, right panel): Antibodies are introduced as in FIG. 8 but bind both to C and Sc prions.

(B, left panel): The clamped puck is agitated gently for, for example, 30 minutes. The puck is inverted and the clamps are opened to allow the contents to drain through coarse mesh 6. Antibody-coated capture beads remain behind. The beads are now bound to both C and Sc prions.

(B, right panel): The puck is turned through 90 degrees to allow the beads to fall into test tube 10. Proteinase K, optionally together with a releasing buffer as discussed in relation to FIG. 8, is added through side port 7 and the beads and proteinase K are incubated for, e.g. 30 minutes. Proteinase K degrades C prions, leaving only Sc prions.

(C, upper drawing): Test tube 10 is removed from the puck and transferred to a holder. Solution containing proteinase K and Sc prions is removed, leaving the beads behind.

(C, lower drawing): The solution is transferred to a microtitre plate well for further analysis.

FIG. 10: Off-line detection of Sc prions according to the invention via antibodies specific to the Sc prion.

(A, left panel): Arrangement of leukocyte-reduction filter for off-line testing, between blood sample and leukocyte-reduced blood; leukocyte-reduction filter is removed as shown when blood has flown through it. A blood sample is provided input bag 1 and is allowed to flow through open clip 2A into leukocyte-reducing filter 3, then through open clip 2B into output bag 4. Clips 2A and 2B are closed around leukocyte-reducing filter 3. The filter, clips and adjacent tubing are removed.

(A, right panel): Assay cube 5 and septum 6 are connected. Detergent and antibody-coated capture beads are added through septum 6 as shown by arrow 7. Clip 2A is opened to allow the detergent and antibody-coated beads to flow into the filter.

(B, left panel): The assembly is agitated for, e.g. 30 minutes (with both clips 2A and 2B closed), to allow the antibodies to bind to Sc prions.

(B, right panel): The assembly is inverted and clips 2A and 2B are opened. The contents of the filter drain through assay cube 5.

(C, left panel): The assay cube is turned through 90 degrees to allow the beads to fall into the V-shaped portion.

(C, right panel): Prion-releasing buffer (see FIG. 8 for details) is introduced through the septum and incubated for, e.g. 5 minutes with the beads. Sc prions are released.

(D, upper drawing): Prion-containing solution is extracted from the assay cube, leaving the beads behind (top drawing).

(D, lower drawing): Prion-containing solution is then transferred to a mitrotitre plate well for further analysis (bottom drawing).

### DETAILED DESCRIPTION OF THE INVENTION

Blood-Borne Agents

According to the invention, any blood-borne agent present in, or on the surface of, white blood cells, or present in or on the surface of platelets may be tested for. Preferred agents are disease agents, i.e. agents present in blood at cause, or are associated with disease. It is particularly preferred to test directly for the infectious agent itself, rather than some derivative or marker associated with the infectious agent It is particularly preferred to test for prion agents. A particularly preferred agent to test for is the scrapie (Sc) prion, which is associated with new variant Creutzfeldt-Jakob disease (vCJD). Thus, the invention provides a method of testing for vCJD in particular.

Concentration of White Blood Cells and/or Platelets

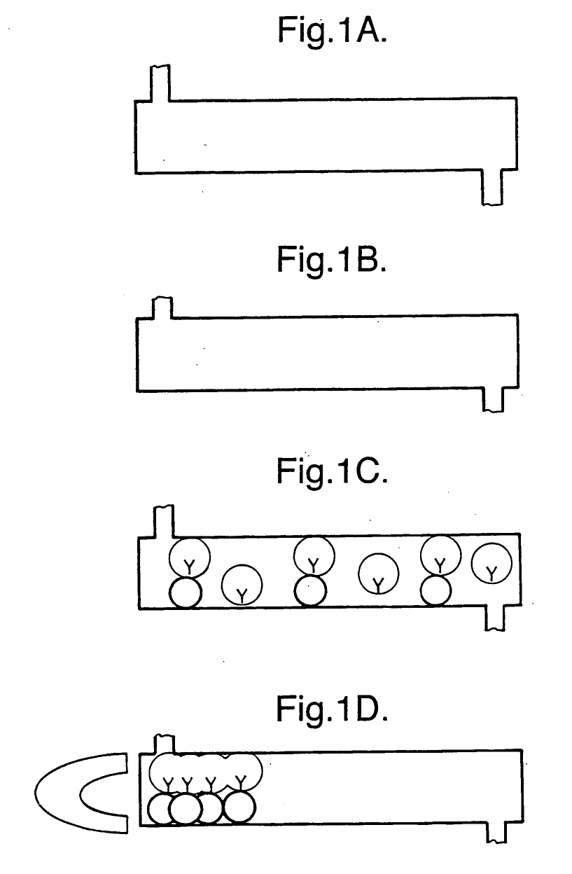

Concentrated white blood cells and/or platelets for testing according to the invention may be obtained by any suitable method. Preferably, they will be obtained by leuko-filtration using a leuko-reducing filter. This can be done as part of the standard procedure in a blood donation program. With reference to FIG. 1, a leuko-reducing filter is used to remove white blood cells from whole blood or fractions of blood containing white blood cells. In a preferred embodiment of the invention, the leuko-reducing filter serves directly as the basis for the testing methods of the invention. This is advantageous as it is convenient to the operator of the method, it requires no additional equipment and it makes use of what would have always been a waste product. Alternatively, however, a concentration, preferably leuko-reducing filtration, step can be carried out especially for the purpose of generating a white blood cell-enriched sample on which to carry out the method of the invention. In that case (see FIG. 1, right-hand side) a sample will preferably be removed for the purpose of the method of the invention, with a second leuko-reducing filtration step to remove further white blood cells, if desired, to ensure blood safety. Alternatively (if, for example, the method is carried out in the context of a blood donation program that does not routinely require leuko-reducing filtration), the concentration step to generate a sample for the method of the invention may be the only step in which white blood cells are removed.

In the context of a blood donation program, it is preferred that each blood donation will be processed individually according to the method of the invention. Therefore, it can be known exactly which blood donations do and do not contain the agent being tested for.

The test sample may be enriched in white blood cells and/or platelets to any extent sufficient to give a large enough amount of white blood cells and/or platelets for reliable testing. Naturally, the amount of white blood cells and/or platelets required will vary with the concentration of the test agent, the sensitivity of the task and a degree of certainty which is required. Therefore the degree of white blood cell and/or platelet concentration needed will vary from case to case, but is within the capacity of the skilled person to judge.

In general, a blood donation has a volume of about 0.5 litres and the sample to be tested will comprise 20% or more of the white blood cells and/or platelets present in said 0.5 litre volume. More preferably, the sample will comprise 50% or more, 75% or more, 90% or more, 95% or more, 99% or more, 99.9% or more, 99.99% or more, or 99.999% or more of said white blood cells and/or platelets.

Put another way, the sample will preferably be enriched by a factor of at least 2, at least 5, at least 10, at least 100, at least 1000 or at least 10000 in white blood cells and/or platelets compared to the whole blood.

Put another way, and especially in situations where the blood sample is not from a blood donation, the sample will preferably be enriched to the extent that it comprises an amount of white blood cells and/or platelets equivalent to 20% or more, 25% or more, 30% or more, 50% or more, 75% or more, 90% or more, 95% or more, 99% or more, 99.9% or more, 99.99% or more, or 99.999% or more of the white blood cells and/or platelets present in a standard 0.5 litre sample of normal human blood.

The number of white blood cells and/or platelets in a blood sample may also vary from individual to individual, or from time to time. For example, individuals suffering from certain conditions may have more or less white blood cells than normal. A skilled person will also take this into account when determining what degree of sample size and enrichment is necessary.

Leuko-reducing filters are known in the art and commercially available, e.g. from Baxter, Pall and HemaSure. Any suitable filter can be used, and it is preferred to use the type of filter that is normally used in a given blood donation program.

It is preferred to carry out the methods of the invention on human blood, though embodiments can also be envisaged in which animal blood, e.g. cow, pig, sheep, goat, horse or dog blood is tested.

Testing Methods

As discussed above, the methods of the invention can be used to test for any blood-borne agent found in or on the surface of white blood cells and/or platelets. Preferably, the agent tested for will be one that is localised partially or completely in or on the surface of white blood cells and/or in or on the surface of platelets, such that white blood cells and/or platelets are a more concentrated source of the agent than, for example, whole blood or plasma. Typically, in the case of white blood cells, the white blood cells will be lysed during the testing process, so the agent may be one which is found inside white blood cells, i.e. in the cytoplasm, or one that is located on the surface of the white blood cells or both. In this connection, it should be noted that white blood cells of individuals infected with vCJD also contain the “normal” C prion in addition to the undesirable Sc prion which is characteristic of vCJD.

In order to determine the presence or absence, or amount or concentration, of the test agent, any suitable test method may be used. Based on his/her general knowledge of the art and on the disclosure herein, the skilled person will be able to decide appropriate test methodologies for any given test agent. Preferred testing methodologies are discussed below in the context of the Sc prion characteristic of vCJD.

Typically, testing for the Sc prion is carried out by a process comprising: (a) lysing said white blood cells to release prions contained in them; (b) contacting the lysate with a reagent or combination of reagents that discriminates between Sc prions present in samples infected by vCJD and C prions naturally present in samples uninfected by vCJD; and (c) visualising or otherwise detecting said Sc prions.

In one preferred embodiment, plasminogen is used to discriminate between Sc and C prions (Fischer M. B. et al, Nature 2000, November 23, 408, 6811, pp479-83). Plasminogen binds to Sc prions but not C prions. Any suitable method may then be used to separate the plasminogen-bound Sc prions from the unbound C prions, or to detect the Sc prion without separation. Preferably, the plasminogen-bound Sc prions will be separated from the unbound C prions. More preferably, magnetic separation techniques will be used. In this case, the lysate is contacted with plasminogen bound to a magnetic substrate, e.g. magnetic beads. These can then be separated from other components, including unbound C prion, using a magnet or electromagnet. The plasminogen-bound Sc prion can be visualised in any suitable way.

One preferred visualisation technique is to use a labelled antibody, which binds to both Sc and C prions, then to separate Sc and C prions, e.g. by magnetic methods as discussed above, then to visualise the Sc prions via the label attached to the antibody. Any suitable label may be used, one preferred label is fluorescein. Others include enzymes, radioactive isotopes, chromophores, dyes, colloidal gold and colloidal carbon.

Preferably, plasminogen/magnetic beads and fluorescein-label antibodies will be used in conjunction, as shown in FIGS. 3 to 6 and discussed in the description of those figures.

Another preferred technique is to use proteinase K to discriminate between c and Sc prions. Proteinase K degrades (digests) C prions but not Sc prions. Therefore, the lysate can be contacted with proteinase K, which will degrade the C prions, leaving the Sc prions. The Sc prions may then be visualised by any suitable method, e.g. by binding to labelled antibodies as discussed above.

The proteinase K will degrade all or substantially all the C prions. For example, it may degrade at least 90, 95, 99, 99.9, 99.99% or 99.999% of the C prions whilst still leaving enough Sc prions for a reliable detection to be carried out.

In another preferred embodiment, an antibody specific to the Sc form of the prion is used to discriminate between the Sc and C prions. Appropriate detection techniques, e.g. as described herein, are then used to visualise the bound antibody/Sc prion complex in order to effect detection of the Sc prion.

Applications of the Invention

Most preferably, the invention is applied in the context of a blood donation program. Donors give blood and samples and, preferably, every sample, is tested according to methods of the invention. Tests for different blood-borne agents may be carried out on the same white blood cell- and/or platelet-enriched sample, or on sub-sets of the sample if enough white blood cells and/or platelets are present. Thus, different techniques according to the invention can be combined.

The invention may also be applied in any context that requires the test of blood and enables the provision of white blood cell-enriched and/or platelet-enriched samples.

For example, the invention may be applied to the detection of blood-borne agents in a diagnostic context by leukophoresis. Leukophoresis is similar to dialysis, in that an open loop is set up, that the patient's blood flows out of the body, through an apparatus and then back into the body. Outside the body, white blood cells are removed, providing a white blood cell-enriched sample outside the body and returning white blood cell-depleted blood to circulation. The invention can be applied to carry out diagnostic tests on such white blood cell-enriched samples removed by leukophoresis.

Also, the invention can be applied in the context of livestock slaughter. This raises issues which are in some ways similar to those faced in blood donation programs. In blood donation programs, the aim is to prevent infected blood from reaching the population that requires blood transfusions. In the slaughterhouse, the aim is to prevent diseased meat from reaching the consumer. This is of particular importance at the present time, in view of the widespread infection of cattle by BSE over the past years, and also because of other public health issues. BSE and scrapie in sheep are of course prion-mediated diseases, and concentration of white blood cells will also benefit testing for prion-mediated diseases of livestock. The methods of the invention can therefore be applied by draining blood from the slaughtered animal, concentrating white blood cells and testing as discussed herein. Similarly, such procedures could be used to monitor the health of animals on farms prior to slaughter, and in programs to contain the spread of disease.

In any of the embodiments of the invention, testing may be qualitative or quantitative. In qualitative tests, the aim is to determine the presence or absence of a test agent. In quantitative tests, the amount or concentration of the test agent is measured. It is envisaged that many applications will be presence/absence tests, in the sense that the information provided by the test will be interpreted as determining the presence or absence of the test agent. However, the test method will generally be one which does produce a numerically quantifiable result, e.g. via measurement of fluorescence or densitometry, even when interpreted in a presence/absence manner.

Kits

The invention also provides kits for carrying out the methods of the invention. Such kits comprise a reagent or reagents capable of detecting the test agent in question. Preferably, they also comprise a leuko-reducing filter “puck” unit. Desirably, the reagents and the filter will be presented as one component so that testing takes place automatically as the white blood cells and platelets are collected by the filter. For example, the detection reagents may be incorporated into the filter unit. In one possible embodiment, the results of the detection can then be visualised directly through a window in the filter unit. Alternatively, the reagents may be washed out for visualisation.

In addition, kits may contain a reader capable of visualising the detection reagents, e.g. a fluorescence monitor or densitometer.

Testing Apparatus

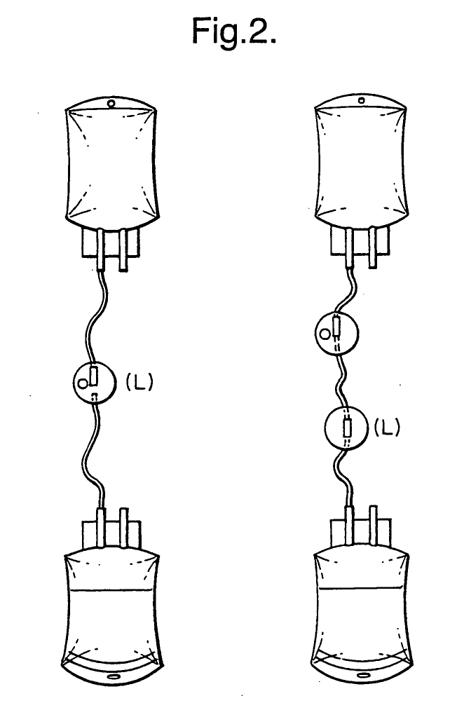

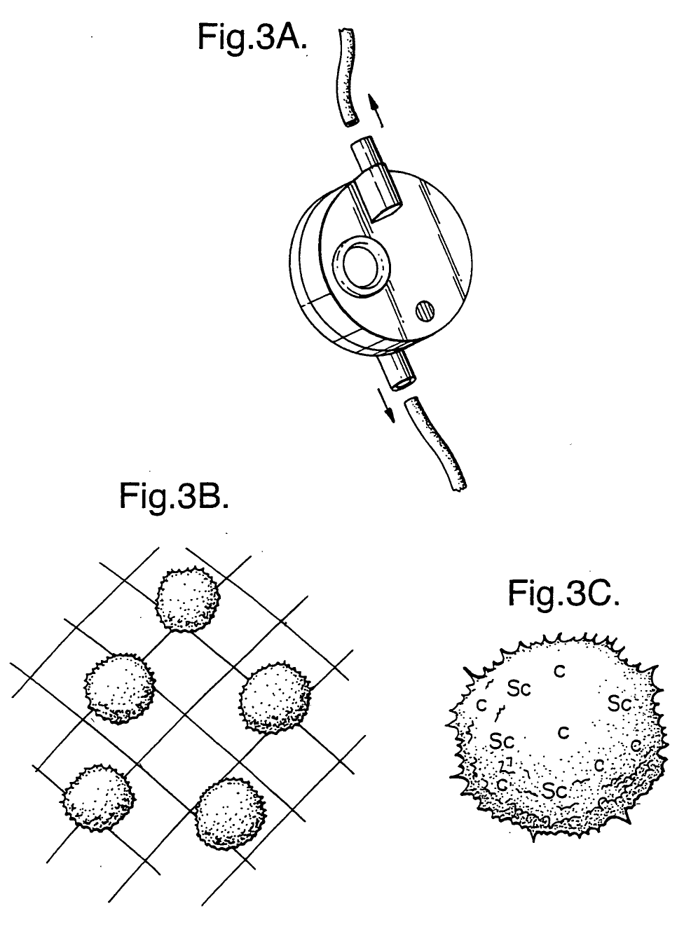

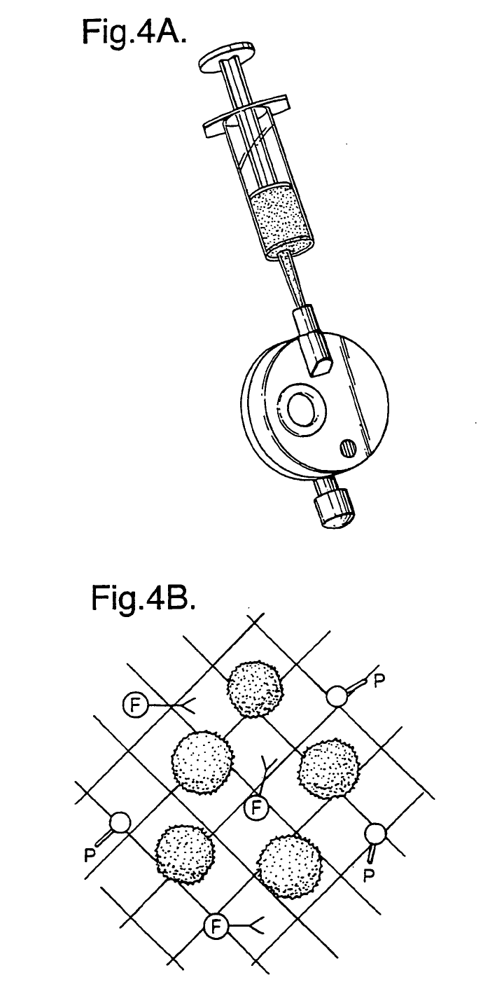

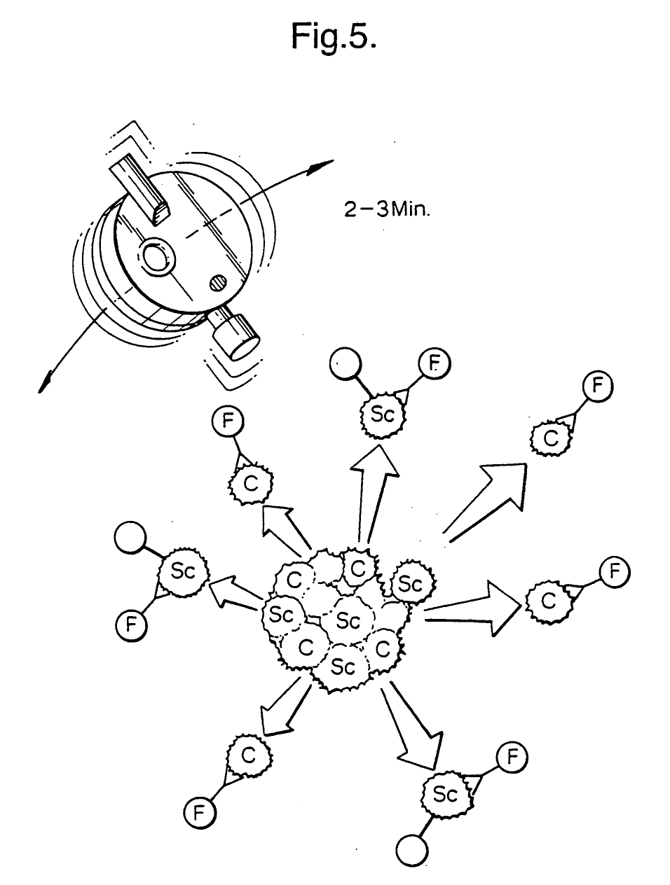

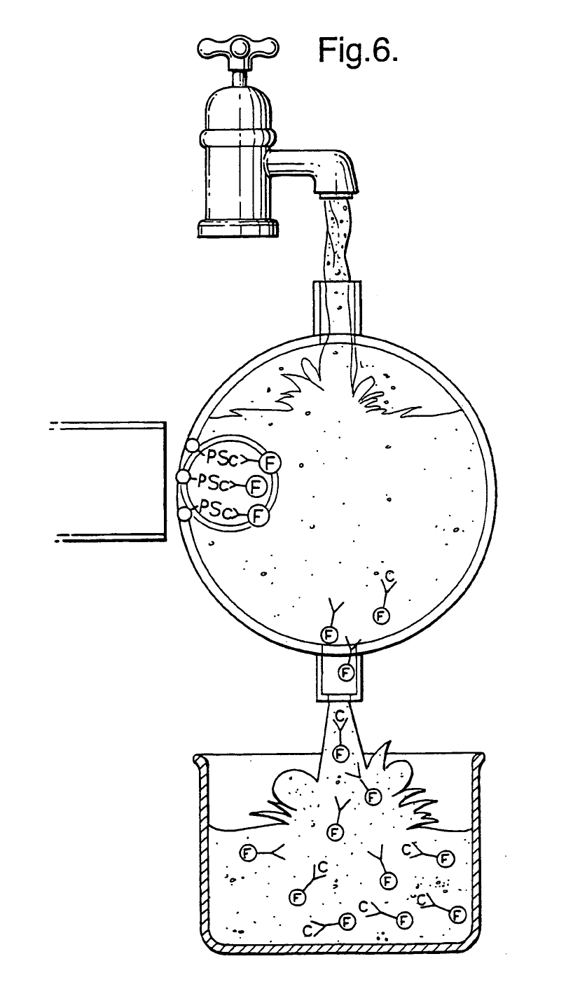

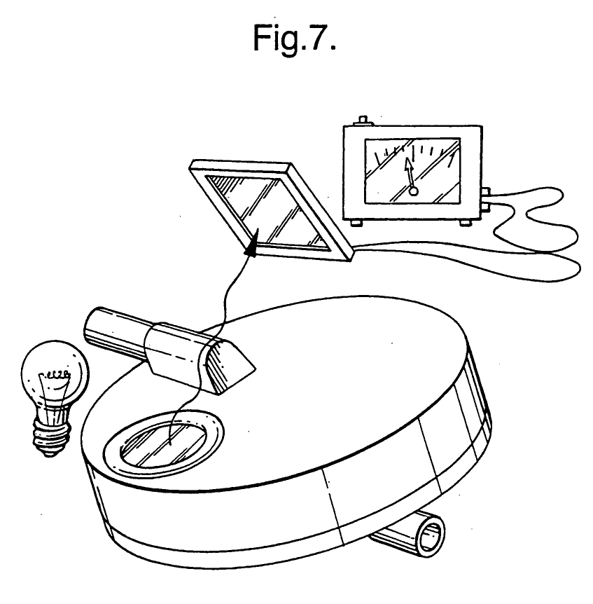

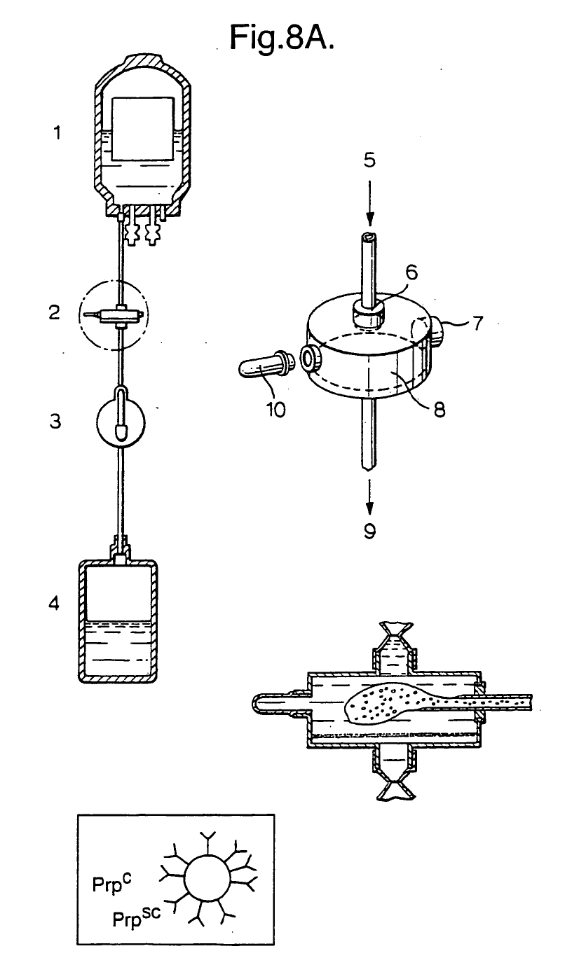

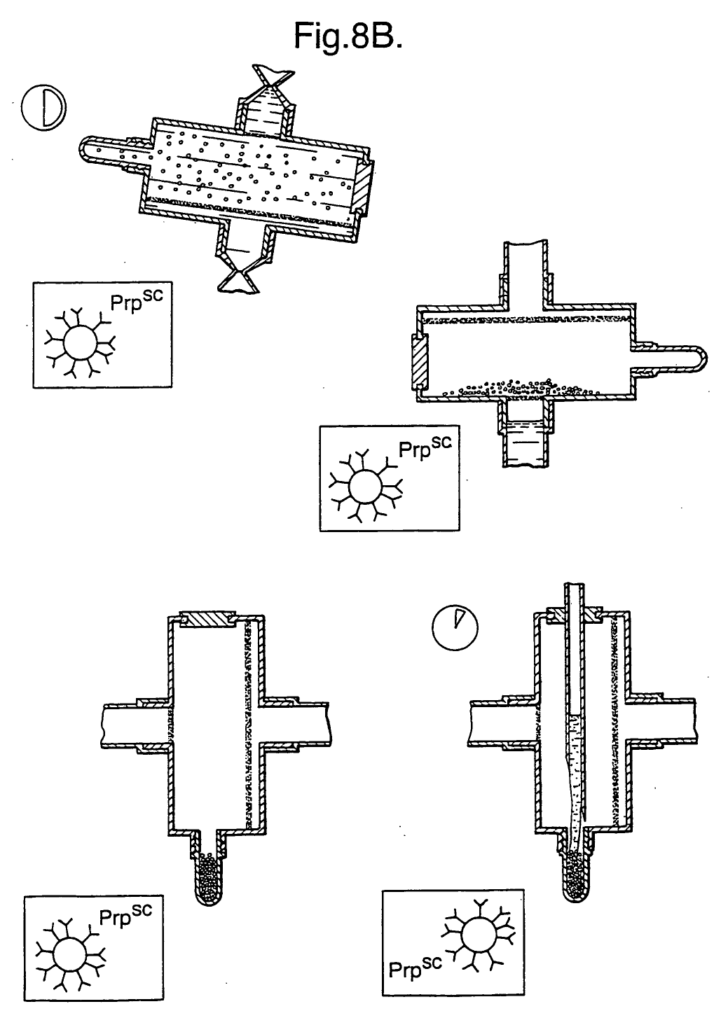

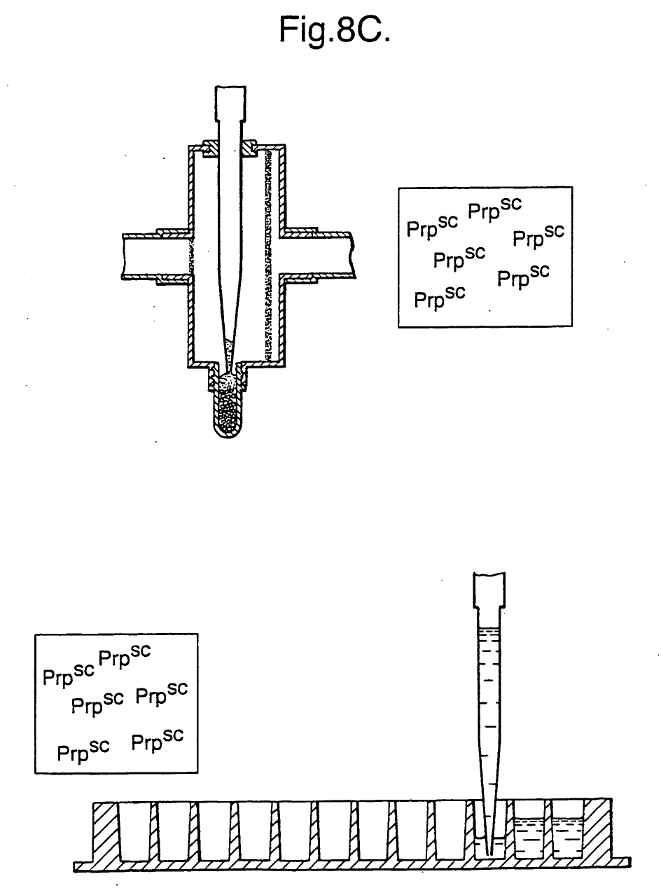

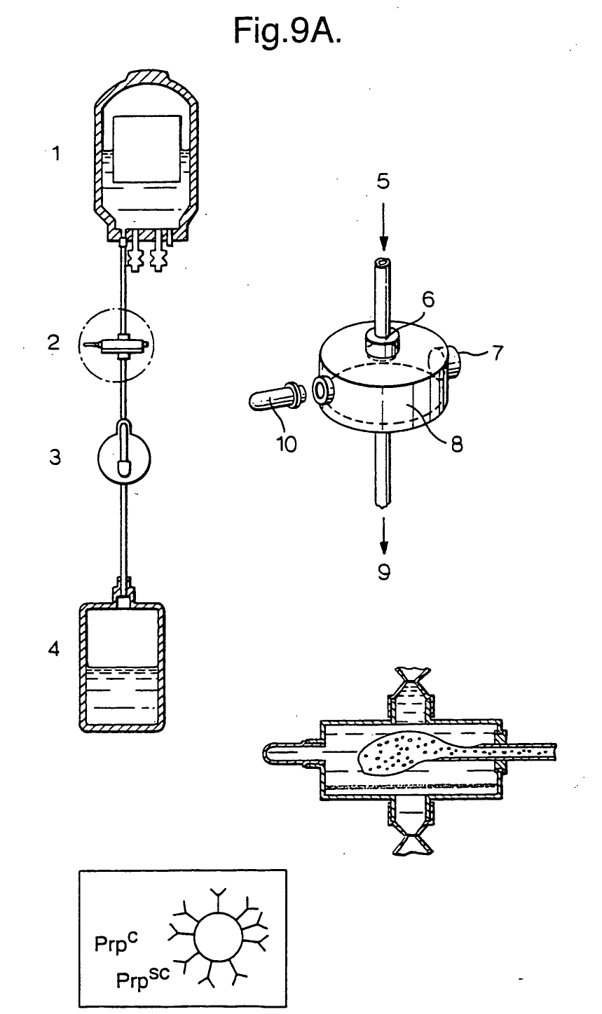

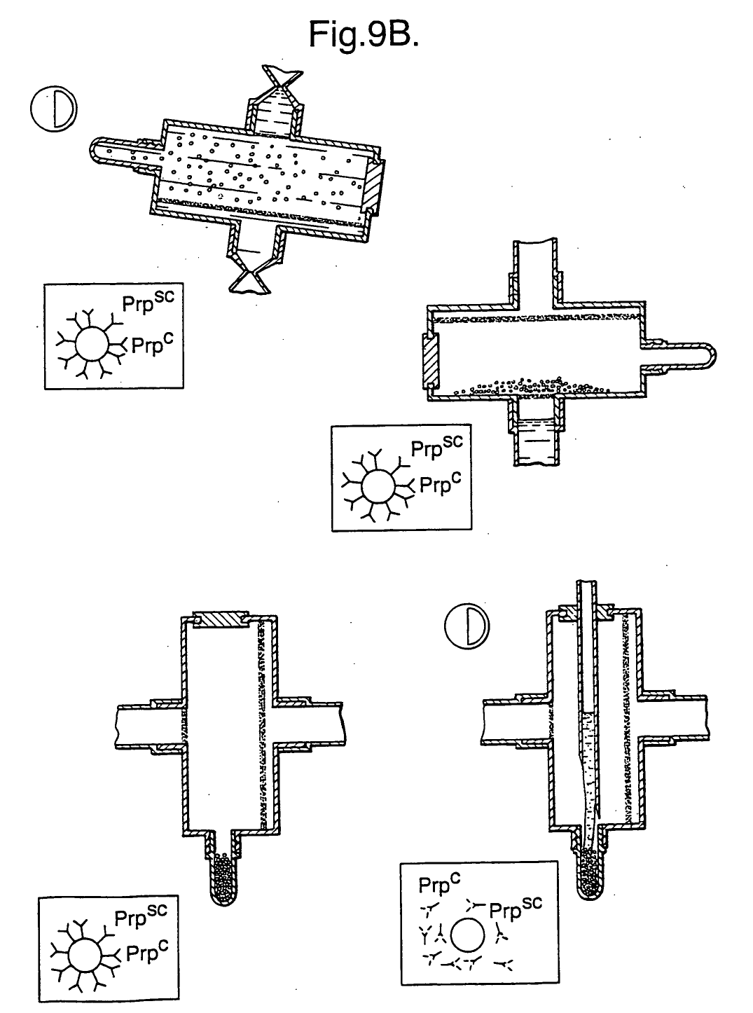

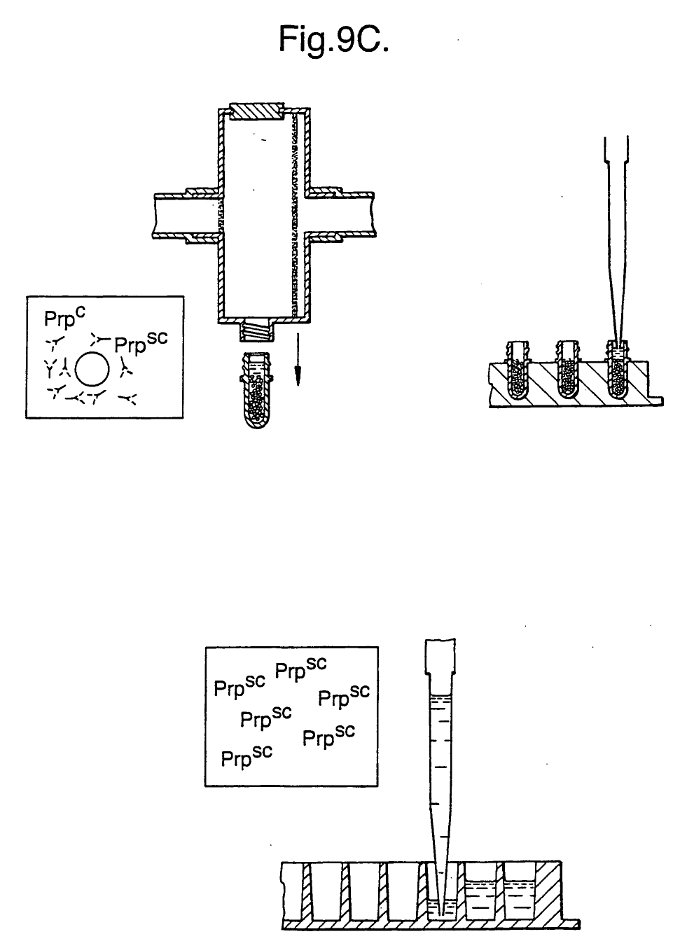

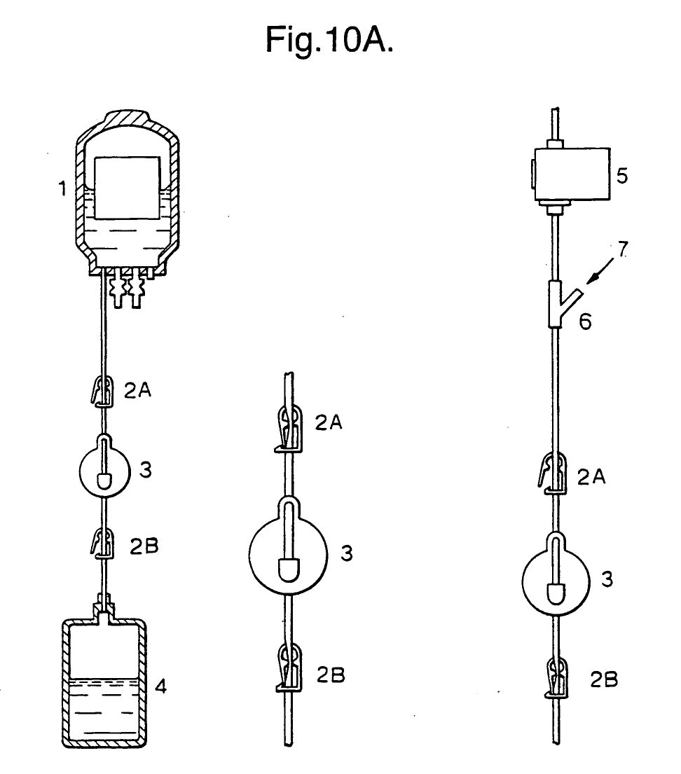

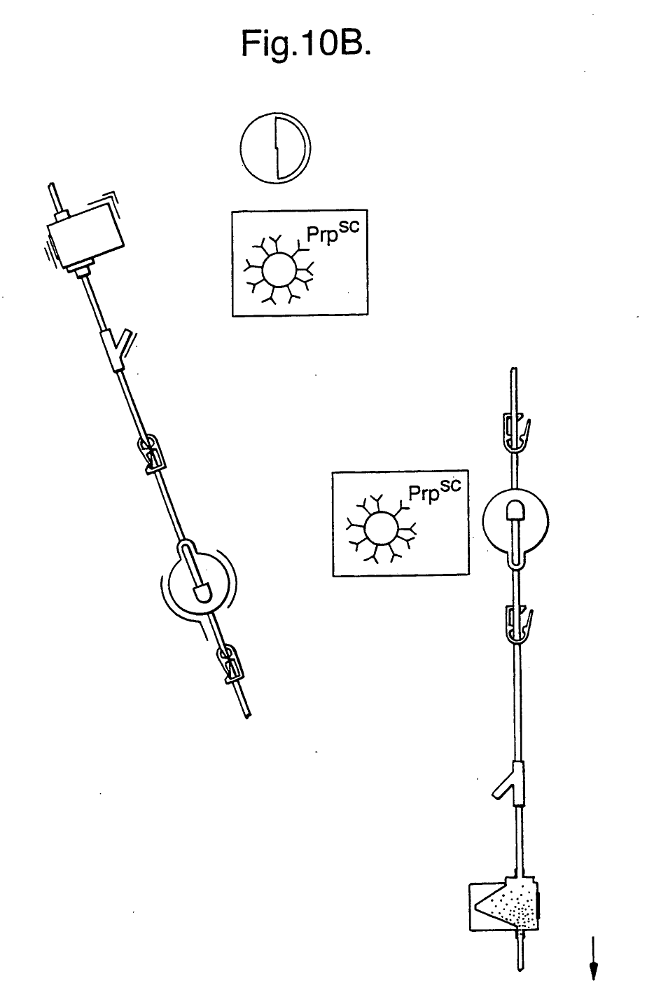

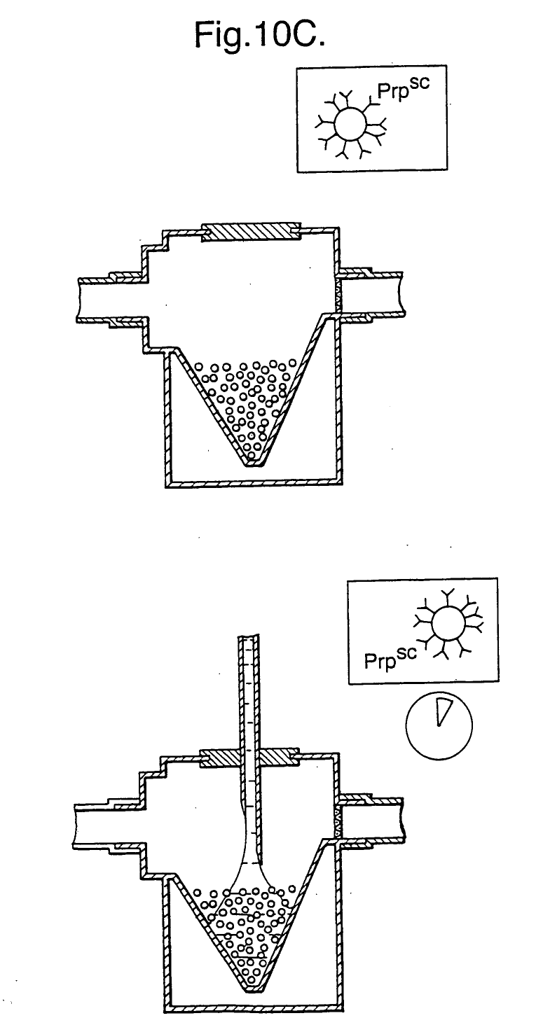

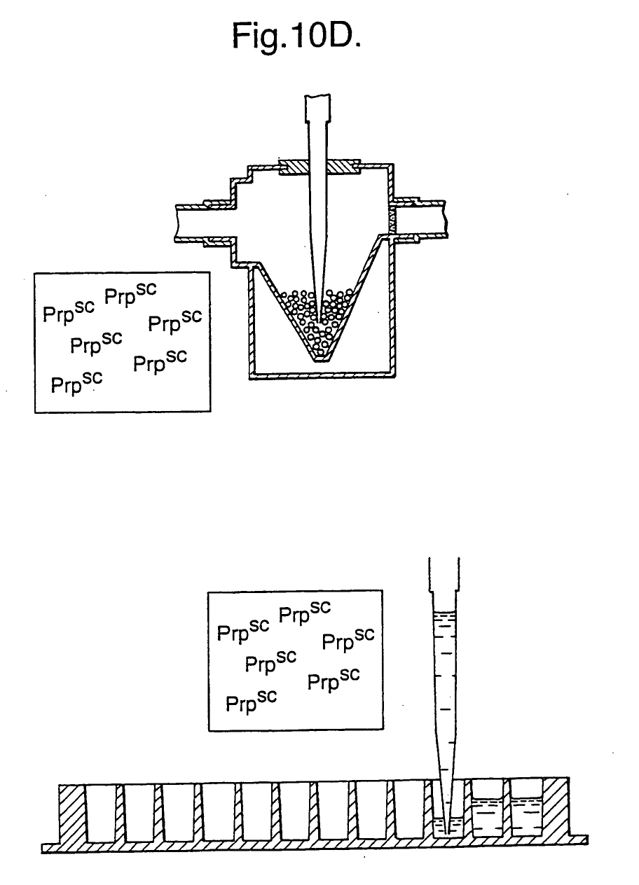

Testing methods of the invention can be carried out using standard apparatus, in particular standard leukocyte-reduction filters. Some possible embodiments are shown in FIGS. 2 and 8 to 10. As will be seen from those Figures, a leukocyte-reduction filter will be provided below the whole blood sample, e.g. blood donation. Blood flows through it, and white blood cells and platelets will become concentrated. The blood then flows into the collection vessel at the bottom for further processing or storage.

Two general arrangements of the apparatus are envisaged, one for in-line testing and one for off-line testing.

In-line testing is shown in FIG. 2 and FIGS. 8/9. Here, the test is carried out in an assay “puck” between the input bag and the leukocyte-reducing filter.

Off-line testing using the leukocyte-reducing filter itself is shown in FIG. 10. This has the advantage that no re-validation is required for existing blood filters because it is clear that there is no impact on the performance of the filters for their existing leuko-reducing purposes. If a technique that might have an impact on such performance is used, then it needs to be proved to the regulatory authorities that the performace of the filter remains unchanged. This creates a cost burden that is desirably avoided.

The embodiments of FIGS. 8 to 10 are described with reference to testing for Sc prions in the context of tests for vCJD. However, the skilled person will appreciate that the same apparatus can also be used to test for other blood-borne agents according to the invention, and that suitably adapted procedures can readily be devised. Similarly, FIGS. 8/9 show testing both with antibodies specific to the Sc prion and with antibodies that bind both Sc and C prions, with subsequent digestion of C prions by proteinase K whilst FIG. 10 specifically shows only the first method. However, the second (proteinase K) method can of course be applied in an off-line apparatus as well.

More generally, a skilled person would appreciate that the apparatus of FIGS. 2 and 8 to 10 can be adapted to test for any blood-borne agent according to the invention.

1. A method of testing for a blood-borne agent present in or on the surface of white blood cells or in or on the surface of platelets, comprising: (a) concentrating white blood cells and/or platelets from whole blood or a fraction of blood containing white blood cells by means of a leuko-reducing filter, thereby providing a blood-derived sample enriched in such white blood cells and/or platelets;
and (b) testing for the presence or absence, or amount or concentration, of said agent in said sample.

2. A method according to claim 1 wherein said sample is a sample derived from human blood.

3. A method according to claim 1 wherein said sample is derived from a blood donation.

4. A method according to any one of the preceding claims wherein said sample has been enriched to the extent that it comprises an amount of white blood cells and/or platelets equivalent to 20% or more of the white blood cells and/or platelets present in a 0.5 litre sample of normal human blood.

5. A method according to claim 3 wherein said sample has been enriched to the extent that it comprises 20% or more of the white blood cells and/or platelets present in said donation.

6. A method according to claim 4 or 5 wherein said sample comprises 50% or more, 75% or more, 90% or more, 95% more, 99% or more, 99.9% or more, 99.99% or more or 99.999% or more of said white blood cells.

7. A method according to any one of the preceding claims wherein the presence or absence of the agent is tested for.

8. A method according to any one of claims 1 to 6 wherein the amount or concentration, of said agent in said sample and a quantitative measurement of said amount or concentration is obtained.

9. A method according to any one of the preceding claims wherein said agent is a disease agent.

10. A method according to any one of the preceding claims wherein said agent is a prion agent.

11. A method according to claim 10 wherein said prion agent is associated with a prion-mediated disease.

12. A method according to claim 11 wherein said prion agent is the scrapie (Sc) prion, said sample is a sample derived from human blood and said prion-mediated disease is new variant Creutzfeldt-Jakob disease (vCJD).

13. A method according to claim 12 wherein testing for the Sc prion is carried out by a process comprising: (a) lysing said white blood cells to release prions contained in them; (b) contacting the lysate with a reagent or combination of reagents that discriminates between Sc prions present in samples infected by vCJD and C prions naturally present in samples uninfected by vCJD; and (c) visualising or otherwise detecting said Sc prions.

14. A method according to claim 13 wherein an antibody specific to the Sc prion is used to discriminate between Sc and C prions.

15. A method according to claim 14 wherein plasminogen, which binds to Sc prions but not C prions, is used to discriminate between Sc and C prions.

16. A method according to claim 15 wherein said plasminogen is attached to a magnetic substrate that enables separation of the plasminogen-bound Sc prions from the unbound C prions.

17. A method according to any one of claim 14 to 16 wherein Sc prions, and optionally the C prions are also contacted with a labeled antibody that binds both Sc and C and the label is used to visualise antibody-bound Sc prions after separation of Sc and C prions.

18. A method according to claim 17 wherein proteinase K is used to degrade all or substantially all the C prions present leaving Sc prions that can then be visualised or otherwise detected.

19. A method according to any one of the preceding claims wherein concentration is carried out in one step-using a leuko-reducing filter and no further removal of white blood cells is required for blood transfusion purposes.

20. A method according to any one of claims 1 to 18 wherein further removal of white blood cells is carried out subsequent to removal of the white blood cells on which the testing method is to be carried out.

21. Use of a blood-derived sample enriched in white blood cells and/or platelets by means of a leuko-reducing filter in a method of testing for a blood-borne agent present or suspected of being present in or on the surface of said white blood cells and/or platelets.

22. Use according to claim 21 wherein said method is as defined in any one of claims 1 to 18.

23. A kit for carrying out a testing method according to any one of claims 1 to 20 comprising a reagent or reagents capable of detecting said agent and a leuko-reducing filter unit, and optionally a reader capable of visualising the reagent or reagents following detection of the test agent.

24. A kit according to claim 23 wherein said reagent or reagents are incorporated into said filter unit.
